(19) United States
(12) Patent Application Publication
(10) Pub. No.: US 2018/0082033 A1
(43) Pub. Date: Mar. 22, 2018

(54) METHOD AND APPARATUS FOR CONTROLLING AN ARTIFICIAL RESPIRATORY DEVICE VIA A NETWORK

(71) Applicant: AT&T Intellectual Property I, L.P., Atlanta, GA (US)

(72) Inventors: Sheldon Kent Meredith, Roswell, GA (US); William Cottrill, Canton, GA (US); Biren Parekh, Cumming, GA (US)

(21) Appl. No.: 15/273,021

(22) Filed: Sep. 22, 2016

(51) Int. Cl. G06F 19/00; A61M 16/00

(52) U.S. Cl. CPC G06F 19/3406; A61M 16/0057; A61M 16/0051; G06F 19/327; A61M 2205/3584; A61M 2230/63; A61M 2230/005; A61M 2205/3561; A61M 2205/3553

(57) ABSTRACT

A method and apparatus for controlling an artificial respiratory device are disclosed. For example, the method detects a change in an activity level of a user based on data that is gathered from at least one sensor that is located in a vicinity of the user, detects a need for a change to a setting of at least one parameter of an artificial respiratory device associated with the user responsive to the change in the activity level of the user being detected, and changes the setting of the parameter of the artificial respiratory device in accordance with the activity level of the user.

The present disclosure relates to a method and apparatus for controlling an artificial respiratory device via a communications network, e.g., a communications network of a network service provider, a local area network, and the like.

### BACKGROUND

Severe respiratory issues affect millions of people around the world. The respiratory issues may be due to cardio-pulmonary obstructions, emphysema, lung cancer, tuberculosis, severe bronchitis, Amyotrophic Lateral Sclerosis (ALS), spinal damage, obstructive sleep apnea, under-developed lungs (e.g., for infants), asthma, infant apnea (e.g., for infants at risk of sudden infant death syndrome), and the like. An artificial respiratory device may be used to treat a patient with respiratory issues.

### SUMMARY OF THE DISCLOSURE

In one embodiment, the present disclosure teaches a method and apparatus for controlling an artificial respiratory device via a communications network. For example, the method detects a change in an activity level of a user based on data that is gathered from at least one sensor that is located in a vicinity of the user, detects a need for a change to a setting of at least one parameter of an artificial respiratory device associated with the user responsive to the change in the activity level of the user being detected, and changes the setting of the parameter of the artificial respiratory device in accordance with the activity level of the user.

### BRIEF DESCRIPTION OF THE DRAWINGS

The teaching of the present disclosure can be readily understood by considering the following detailed description in conjunction with the accompanying drawings, in which:

FIG. 1 illustrates an example network related to the present disclosure for controlling an artificial respiratory device;

FIG. 2 illustrates a flowchart of an example method for controlling an artificial respiratory device; and

FIG. 3 depicts a high-level block diagram of a computer suitable for use in performing the functions described herein.

To facilitate understanding, identical reference numerals have been used, where possible, to designate identical elements that are common to the figures.

### DETAILED DESCRIPTION

The present disclosure relates to a method and apparatus for controlling an artificial respiratory device via a communications network, e.g., a communications network of a network service provider.

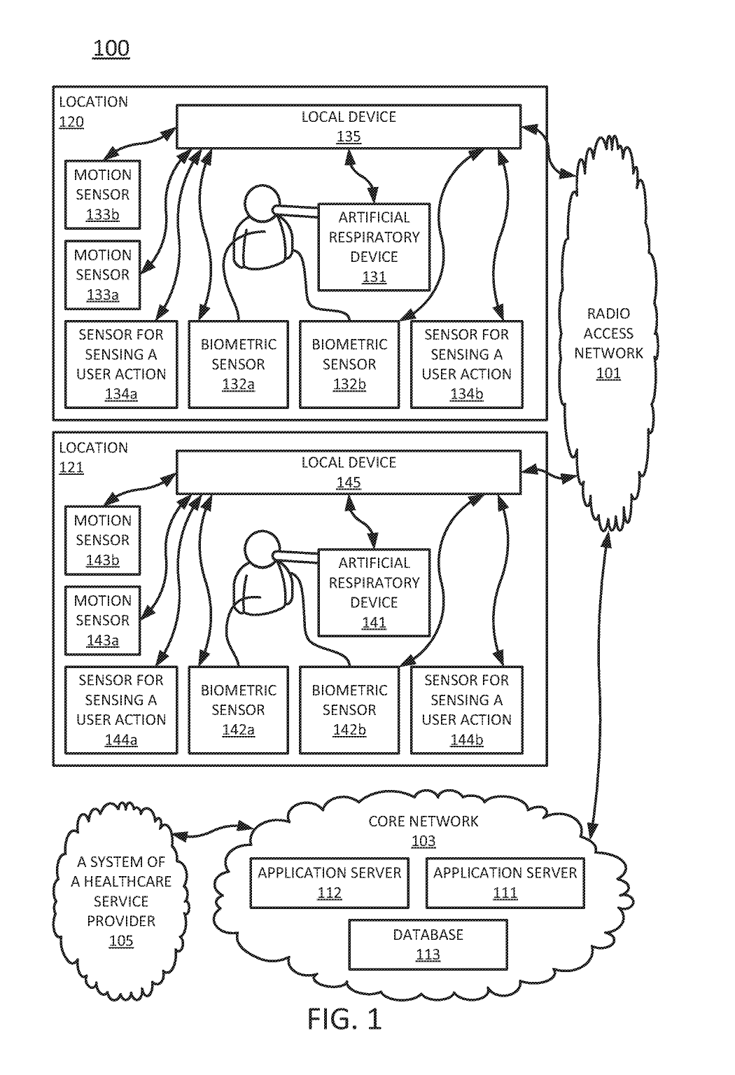

FIG. 1 illustrates an example network 100 related to the present disclosure for controlling an artificial respiratory device. In one illustrative embodiment, the network 100 may comprise a radio access network 101, a core network 103, and a system of a healthcare service provider 105.

The radio access network 101 may comprise cellular or other wireless technologies, e.g., Wi-Fi networks, Long Term Evolution (LTE) networks, 3G, 4G, and 5G networks, and the like. The core network 103 may comprise any number of gateway devices, application servers, routers, switches and databases of a network service provider. For example, the core network 103 may comprise a dedicated application server 111 for controlling one or more medical devices or equipment, e.g., an artificial respiratory device in accordance with the present disclosure, an application server 112 for providing a notification, and a database 113. The database 113 may be used for storing data, e.g., a list of users (e.g., patients), a list of artificial respiratory devices, a list of parameters of artificial respiratory devices, a list of settings of artificial respiratory devices, a list of manufacturers of artificial respiratory devices (e.g., manufacturers from whom data related to parameters and/or settings may be obtained), a list of distributors of artificial respiratory devices, a list of distributors of components of artificial respiratory devices, a list of suppliers of supplies to be used in conjunction with an artificial respiratory device (e.g., suppliers of masks, tubing, filters and the like), data gathered via any number of sensors, a list of recipients of notifications (e.g., nurses, doctors, healthcare service providers, guardians or family members of users, etc.), and the like.

The system of the healthcare service provider 105 may comprise any number and type of hardware systems or servers (not shown) for providing services to the users. For example, a hardware system of the healthcare service provider may comprise one or more servers for obtaining notifications (e.g., notifications regarding settings of artificial respiratory devices, etc.), providing healthcare services via a network, alerting caregivers (e.g., family members) of patients receiving services from the healthcare service provider, etc.

In one embodiment, the example network 100 also illustrates locations 120-121. Each of the locations 120-121 comprises a physical location (e.g., a home, a nursing home, a hospital, a room within the home or any medical facilities, and the like) at which a user of an artificial respiratory device is located. The location 120 comprises an artificial respiratory device 131 for assisting in the breathing process of a patient (broadly a ventilator, a breathing machine, a respirator, and/or a medical device for providing respiratory treatment to a patient), biometric sensors 132a-132b, motion sensors 133a-133b, sensors 134a-134b for sensing a user action, and a local device 135. Similarly, the location 121 comprises an artificial respiratory device 141, biometric sensors 142a-142b, motion sensors 143a-143b, sensors 144a-144b for sensing a user action, and a local device 145.

The artificial respiratory devices 131 and 141 may comprise any appropriate type of medical device for providing respiratory (e.g., breathing) assistance to a user. For example, a user who is unable to breathe completely on his/her own may be assisted by the artificial respiratory device. An artificial respiratory device may comprise an artificial respirator, a trilogy machine, an airway pressure machine, a ventilator, and the like. The artificial respiratory device may also be referred to as an artificial breathing machine.

An airway pressure machine refers to a type of artificial respiratory device that applies air pressure to a lung of a user to keep the airways of the user open. In one embodiment, the airway pressure machine comprises a Continuous Positive Airway Pressure (CPAP) machine. CPAP refers to a form of positive airway pressure ventilator which applies air pressure throughout a breathing cycle to keep the airways continuously open. For example, the CPAP may apply the positive air pressure on a continuous basis using an air flow to generate the air pressure. In one embodiment, the airway pressure machine comprises a Positive End Expiratory Pressure (PEEP) machine. PEEP refers to a form of a positive airway pressure ventilator that applies positive air pressure only at the end of an exhalation.

The biometric sensors 132a-132b and 142a-142b may comprise any appropriate type of device for sensing biometric information of a user. For example, a biometric sensor may be for sensing at least one of: a blood pressure, an oxygen level in the blood of a patient, a pulse rate, a temperature, and so on.

The motion sensors 133a-133b and 143a-143b may comprise any appropriate type of device for sensing a motion of a user. For example, a motion sensor may be for sensing an eye movement of a user, for sensing a head movement of a user, for sensing when the user moves a limb of the user, and the like.

The sensors 134a-134b and 144a-144b for sensing a user action may comprise any appropriate type of device for sensing when a predetermined user action is performed by a user. In one embodiment, an example list of predetermined user actions that are to be sensed may be determined by a healthcare service provider, a caregiver, the network service provider, etc. For example, the healthcare service provider may determine a priori that the user action comprises pressing a buzzer to call a nurse, adjusting a bed setting of an electric bed, opening and/or closing electric window blinds, changing a setting of an entertainment device, e.g., changing a television station via a remote control, and turning a light on or off, and the like.

In one embodiment, the list of predetermined user actions may be based on a condition of the user. For example, suppose a first user is in a coma and a second user is suffering from sleep apnea. The second user may move his/her limb while changing his/her sleeping position. In contrast, the first user may not move much. In one example, for the first user, the list of predetermined user actions may include any perceivable movements of the user. However, for the second user, the list of predetermined user actions may not include moving a limb, unless the location of the user changes significantly.

In one example, the local device 135 comprises a hardware electronic device in the location 120 (i.e., in the vicinity of a user of the artificial respiratory device 131) with a capability to relay signals and/or notifications between the core network 103 and the location 120, via the radio access network 101. Similarly, the local device 145 comprises a hardware electronic device in the location 121 (i.e., in the vicinity of a user of the artificial respiratory device 141) with a capability to relay signals and/or notifications between the core network 103 and the location 121, via the radio access network 101. For example, the local device 135 or 145 may comprise a Wi-Fi hotspot device, a mobile hotspot device, a server, a router, etc.

In one embodiment, the artificial respiratory device 131 or 141 may be equipped with wireless communication capabilities. In one embodiment, any number of the various sensors, e.g., the biometric sensors 132a-132b and 142a-142b, the motion sensors 133a-133b and 143a-143b, the sensors 134a-134b and 144a-144b for sensing a user action, etc., are equipped with wireless communication capabilities.

In one embodiment, the artificial respiratory device 131 or 141 may be a device that communicates via a machine-to-machine (M2M) communications interface. For example, an artificial respiratory device may communicate with a server via an M2M communications interface. In one embodiment, any number of the various sensors, e.g., the biometric sensors 132a-132b and 142a-142b, the motion sensors 133a-133b and 143a-143b, the sensors 134a-134b and 144a-144b for sensing a user action, etc., may also communicate via an M2M communications interface. For example, a sensor may collect data and report to a server via an M2M interface. It is to be understood that the various sensors and artificial respiratory devices depicted in FIG. 1 are only examples and not intended to limit the scope of the present disclosure.

As described above, a user with a respiratory issue may be treated using an artificial respiratory device that applies air pressure. For example, a CPAP machine may be used to apply positive air pressure on a continuous basis (broadly “continuously” on a periodic basis, i.e., every five minutes, every ten minutes, every 30 minutes, every hour, and so on). The CPAP machine may have several parameters. For example, the parameters may comprise at least one of: a parameter for a number of respiration cycles per minute, a parameter for a volume of air exchange per respiration cycle, a parameter for selecting whether the CPAP machine forces air into a lung of a user or applies a positive air pressure to assist the user while the user is trying to breathe on his/her own, etc. For instance, the CPAP machine may comprise a sensor for sensing a negative air pressure when the user is trying to breathe. Then, when the negative air pressure is sensed, the CPAP may apply the positive air pressure to assist the user by keeping the airways open while the user is trying to breathe.

The CPAP machine may have settings that are adjustable. For example, a nurse may make an adjustment to at least one parameter of the CPAP machine. For example, a nurse may adjust the parameter for the number of respiration cycles per minute, the parameter for the volume of air exchange per respiration cycle, the parameter for selecting whether the CPAP machine forces air into the lung of the user or applies the positive air pressure to assist the user while the user is trying to breathe on his/her own, etc.

The above examples illustrate that the settings of the CPAP machines are complex and changing such settings require personnel with know how in the operation of the CPAP machines. In addition, the adjustments to the settings may vary based on an activity level of the user. For example, a user who is walking may require more assistance from the CPAP machine as compared to a user who is sleeping and resting. Furthermore, users of artificial respiratory devices may have unpredictable sleep cycles, e.g., due to sleep apnea, discomfort due to illness, etc. For example, suppose a normal sleep cycle for a person with a normal respiratory function comprises a continuous time interval of eight hours during which the person sleeps, followed by a continuous time interval of sixteen hours during which the person stays awake, followed by a next continuous time interval of eight hours during which the person sleeps, and so on. Then, a person with a normal respiratory function having a normal sleep cycle may sleep for eight hours and stay awake for sixteen hours, continuously alternating between the two time intervals in which the person sleeps and stays awake. In contrast, a user who has a sleep cycle that is unpredictable may not adhere to this example normal sleep cycle. Thus, providing an optimal care to a user with an unpredictable sleep cycle may require frequent adjustments to the settings of the CPAP machine serving the user. Thus, providing an optimal care for a user of an artificial respiratory device is a non-trivial matter.

One approach to provide care for the user is to have a qualified nurse make frequent adjustments to the settings of the CPAP machine. However, the frequent adjustments also make it more difficult for the user to sleep for a continuous time interval. For example, the adjustment itself, the opening and/or closing of doors of a room of the user, the turning on/off lights in the room of the user, etc. may cause the user to wake up. Thus, the quality of life of the user is adversely affected by the frequent adjustments on the artificial respiratory device of the user that are made by a nurse.

In one embodiment, in contrast the present disclosure provides a method for controlling an artificial respiratory device via a network. For example, an application server of a network service provider may control the artificial respiratory device of the user based on an activity level of the user. The activity level of the user may be assessed by analyzing data that is gathered from at least one sensor that is located in the vicinity of the user.

In one embodiment, the data may be gathered from the sensor directly. For example, the sensor may be able to communicate via a wireless network, e.g., via an LTE network, a Wi-Fi network, etc. For example, a sensor at location 120 or 121 may communicate with the remotely located application server 111 via the radio access network 101, without involving the local device 135 or 145, respectively.

In one embodiment, the data may be gathered from the sensor via a local device, e.g., a local device 135 or 145. In one embodiment, the local device may receive the data from the sensor in accordance with a predetermined schedule for receiving data from the sensor. For example, the local device may receive data from the sensor every five minutes. In one embodiment, the predetermined schedule for receiving data from the sensor may be based on a type of the sensor. For example, the predetermined schedule for receiving data from a biometric sensor may be different from the predetermined schedule for receiving data from a motion sensor.

In one embodiment, the local device may provide the data to an application server of the network service provider, e.g., AS 111, in accordance with a predetermined schedule for providing the data to the application server. For example, the data gathered from the sensor by the local device 135 or 145 may be aggregated over a time interval of 15 minutes, 30 minutes, one hour, etc. The local device 135 or 145 may then provide the data that is aggregated to the application server 111.

In one embodiment, the network service provider determines the predetermined schedule for causing the data to be provided to the application server. For example, a controller of the network service provider or an application server of the network service provider may transmit the predetermined schedule to the local device. The local device may then forward the data that is aggregated in accordance with the predetermined schedule that is received from the network service provider.

In one embodiment, the method detects a change in the activity level of the user by analyzing the data that is gathered from the sensor that is located in the vicinity of the user. The change in the activity level of the user is detected when the change in the activity level of the user reaches or exceeds a predetermined threshold for the change in the activity level of the user.

In one embodiment, the predetermined threshold for the change in the activity level of the user may be based on a profile of the user. In one embodiment, the profile of the user comprises at least one of: demographic information of the user and a condition of the user. The demographic information of the user may comprise an age of the user, a weight of the user, etc. The condition of the user may be for indicating a health condition of the user. For example, suppose a first user is 90 years old and is not moving much regardless of the time of day and a second user is 20 years old and is being treated for sleep apnea. Then, the predetermined threshold may be different for the first and second users, e.g., the activity levels threshold for the older patient can be set lower than a younger patient, and the like. In another example, suppose a third user is 20 years old, weighs 150 lbs and is being treated for a severe case of pneumonia and a fourth user is 20 years old, weighs 150 lbs and is in a coma. Then, the predetermined threshold may be different for the third and the fourth users based on the differences in the health conditions for which the third and fourth users are being treated, e.g., the activity levels threshold for the patient in a coma can be set lower than a patient not in a coma, and the like. These examples are only illustrative and are not intended to limit the present disclosure since any number of demographic parameters and medical conditions can be used.

When the change in the activity level of the user is detected, the method performs an analysis to determine whether there is a need for changing a setting of at least one parameter of the artificial respiratory device in response to the change in the activity level of the user. In other words, responsive to the change in the activity level of the user reaching a threshold, the method may detect a need for a change to a setting of at least one parameter.

As described above, the artificial respiratory device may comprise at least one of: a parameter for a number of respiration cycles per minute, a parameter for a volume of air exchange per respiration cycle, a parameter for selecting whether the artificial respiratory device forces air into a lung of the user or applies a positive air pressure to assist the user by keeping the airways open while the user is trying to breathe. In sum, any number of the parameters described above may be parameters that are controlled in accordance with the teachings of the present disclosure. Thus, the method determines whether a need for changing a setting of at least one parameter of the artificial respiratory device of the user is detected. For the parameter of the artificial respiratory device for which the need for changing the setting of the parameter is detected, the method changes the setting of the parameter in accordance with the activity level of the user. For example, suppose a need for changing the setting of a parameter for a number of respiration cycles per minute is detected when a user who was previously asleep is currently awake. In turn, the method determines a new setting of the parameter for the number of respiration cycles per minute that is applicable for an activity level of the user when the user is awake, e.g., increasing the number of respiration cycles per minute relative to the number of respiration cycles per minute when the user was asleep.

In one embodiment, the setting of the parameter of the artificial respiratory device of the user is changed by selecting a setting of the parameter from a set of allowable settings of the parameter in accordance with the activity level of the user. In one embodiment, the setting of the parameter is selected from the set of allowable settings at least in part based on the change in the activity level of the user from a previous activity level of the user. For example, suppose for a user who is waking up from a short nap and eye movements are detected but the biometric information (e.g., heartrate) for the time interval during which the user is awake is the same or similar to the biometric information for the time interval during which the user was taking a short nap, then the deviation in the activity level of the user may be considered “small.” In other words, a mere detected change in the activity level may in and of itself not be sufficient to cause a change in setting if the biometric information indicates a lack of a need to change a setting of the parameter. In another example, suppose a user who was previously observed sleeping is observed walking and the biometric information (e.g., heartrate) indicates a significant change, then the deviation in the activity level of the user may be considered “large.” In other words, in this example, a detected change in the activity level may cause a change in setting if the biometric information confirms the need to change a setting of the parameter. The use of the relative terms “small” and “large” are only illustrative and can be quantified for a parameter of the artificial respiratory device to meet a particular deployment requirement.

In one embodiment, the setting of the parameter of the artificial respiratory device of the user is changed by modifying the setting of the parameter on a continuous basis. For example, the activity level of the user may be analyzed continuously, and the setting of the parameter may be selected in accordance with the result of the latest analysis. For example, the setting of the parameter may be based on the newest activity level of the user.

In one embodiment, although the setting of a parameter of the artificial respiratory device of the user can be selectively changed, the change is constricted to a set of allowable settings for the parameter e.g., constricted to a predefined range for the parameter. For example, a parameter related to the volume of air exchange per respiration cycle may be constricted to prevent excessive oxygen level from causing injury to a user. For instance, the appropriate volume of air exchange and oxygen level may depend on the size of the user. To illustrate, for an infant user, the predefined range of the settings of the parameter related to the volume of air exchange per respiration cycle may be constricted to a narrower range as compared to the predefined range of the settings for the same parameter for an adult user. In other words, for the infant user, the set of allowable settings of the parameter related to the volume of air exchange per respiration cycle may be constricted to a narrower range of the settings for the parameter.

In one embodiment, the set of allowable settings for the parameter is constricted to the predefined range of the parameter based on a profile of the user. In one example, the set of allowable settings of the parameter is constricted based on a profile of the user that indicates a demographic information of the user, e.g., an age and/or a weight of the user. In another example, the set of allowable settings of the parameter is constricted based on a profile of the user that includes a health condition of the user, e.g., whether the artificial respiratory device is being used to assist a user who is unable to breathe on his/her own or the artificial respiratory device is being used to assist a user partially breathing on his/her own.

In one embodiment, the method records the setting of the parameter of the artificial respiratory device of the user that is changed. For example, the change to the settings of the parameter may be stored in a database, e.g., database 113.

In one embodiment, the method provides a notification. For example, the notification may comprise the setting of the parameter of the artificial respiratory device of the user that is changed. In one embodiment, the notification may be provided to a system of a healthcare service provider (e.g., a system to be used by a healthcare professional). For example, the healthcare professional may be a doctor, a nurse, a respiratory therapist, and the like. For instance, the healthcare profession may use the notification to detect an improvement or a worsening in a condition of the user, to provide healthcare services to the user, to notify caregivers of the user, and so on.

In one embodiment, the notification may be provided to a system of at least one of: a caregiver, a family member, or a next of kin of the user. For example, the notification may be provided to a mobile device (e.g., a smart phone, a laptop, a computing tablet, etc.) of a family member of the user.

In one embodiment, the notification may be provided to a local device. For example, the local device may be at a nurse station of a hospital, at a nurse station of a nursing home, and so on.

In one embodiment, the notification is provided to at least one server that performs a remote function. In one example, the remote function may be monitoring of the artificial respiratory device. In another example, the remote function may be scheduling a repair and/or maintenance for the artificial respiratory device. In yet another example, the remote function may be scheduling delivery of supplies to be used in conjunction with the artificial respiratory device (e.g., oxygen tank, mask, filter, and the like), and so on. It is noted that the various remote functions may be performed via any number of servers. Thus, the notification may be provided to any number of servers.

It should be noted that the network 100 may include additional networks and/or elements that are not shown to simplify FIG. 1. For example, the radio access network and the core network of FIG. 1 may include additional network elements (not shown), such as for example, base stations, border elements, gateways, firewalls, routers, switches, call control elements, various application servers, and the like. In addition, various types of data may be stored in any number of databases. For instance, various databases, e.g., a database for a list of artificial respiratory devices that are being controlled, a database for parameters and/or settings of parameters of artificial respiratory devices that are being controlled, a database for users associated with each of the artificial respiratory devices in the list of artificial respiratory devices that are being controlled, a database for storing profiles of users, a database for data gathered via at least one sensor, a database for storing biometric data of users associated with the artificial respiratory devices that are being controlled, a database for user actions (e.g., actions to be taken for the patient), a database for storing changes (i.e., modifications in view of various thresholds being reached for each patient) to parameters and/or settings of parameters of artificial respiratory devices that are being controlled, etc., may be provided. In addition, the various types of data may also be stored in a cloud storage. In other words, the network service provider may implement the service for controlling an artificial respiratory device of the present disclosure by storing data in a cloud storage and/or a centralized server.

In one embodiment, the AS 111 is used for implementing the present method for controlling an artificial respiratory device. The AS 111 of the present disclosure is for detecting a change in an activity level of a user based on data that is gathered from at least one sensor that is located in a vicinity of a user, for detecting a need for a change to a setting of at least one parameter of an artificial respiratory device of the user responsive to the change in the activity level of the user being detected, and for changing the setting of the parameter of the artificial respiratory device in accordance with the activity level of the user. In one embodiment, the AS 111 is also for providing a notification that comprises the setting of the parameter that is changed and for recording the setting of the parameter that is changed. In one embodiment, another application server, e.g., AS 112, is for providing the notification and for recording the setting of the parameter of an artificial respiratory device that is changed. In other words, the controlling of the artificial respiratory device, the providing of the notification, and the recording of the setting of the parameter that is changed may be performed by a same application server or by different application servers.

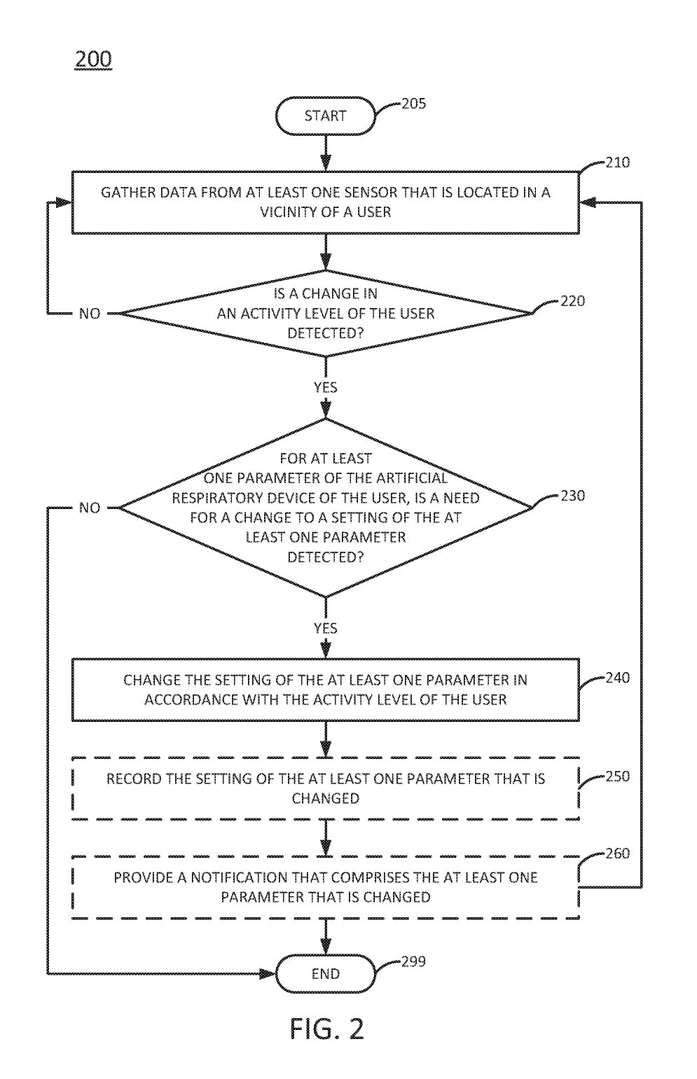

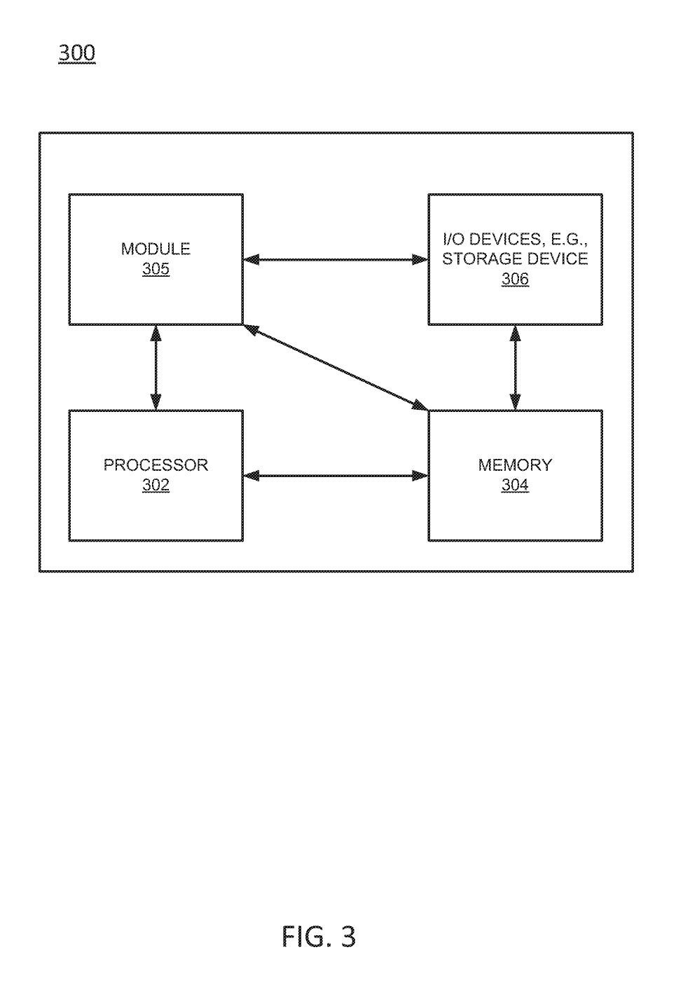

FIG. 2 illustrates a flowchart of an example method 200 for controlling an artificial respiratory device in accordance with the present disclosure. In one embodiment, the method 200 may be implemented in an application server, e.g., an AS 111, or the processor 302 as described in FIG. 3.

The method 200 may be implemented for any number of artificial respiratory devices. For example, the AS 111 may be used for any number of artificial respiratory devices assisting any number of users. For clarity, the flowchart of the example method 200 is described herein for an artificial respiratory device. The method 200 starts in step 205 and proceeds to step 210.

In step 210, the processor gathers data from at least one sensor that is located in a vicinity of a user. For example, the processor may gather data from a biometric sensor, a motion sensor, a sensor for sensing a user action, etc., that is physically located in the vicinity of the user. For instance, the sensor may be in a room in which the user is located.

In step 220, the processor determines whether a change in an activity level of the user is detected based on the data that is gathered in step 210. In one embodiment, the change in the activity level of the user is detected when the change in the activity level of the user reaches or exceeds a predetermined threshold for the change in the activity level of the user. When the change in the activity level of the user is detected as having reached a threshold, the processor proceeds to step 230. Otherwise, the processor returns to step 210.

To illustrate by way of an example, suppose for the user, the activity level of the user reaches the predetermined threshold, when the user is changing his/her position by at least ten degrees and/or moves at least one meter from an earlier detected position. Suppose also the user was previously asleep at an angle of fifteen degrees and is currently awake and is sitting up at an angle of sixty degrees. Then, the processor detects a change of forty-five degrees when the user moves from the angle of fifteen degrees to the angle of sixty degrees. The change in the activity level of the user (i.e., the change of forty-five degrees) reaches or exceeds the predetermined threshold of ten degrees. The detected change in activity can be determined from an analysis of captured images of the user (e.g., comparing time lapsed images of the user on a hospital bed) or data received from an electric bed (e.g., posture or angle setting on the electric bed) used by the user. In one embodiment, a different threshold is set for each individual patient, e.g., a change of forty-five degrees in a bed setting may cause a threshold to be reached for one patient but not for another patient. Thus, the processor proceeds to step 230.

In step 230, the processor determines whether a need for a change to a setting of at least one parameter of an artificial respiratory device of the user is detected. The determination as to whether the need for the change to the setting of the parameter of the artificial respiratory device of the user is made in response to the change in the activity level of the user being detected. In one example, the processor may detect the need for the change to a setting of a parameter for a number of respiration cycles per minute. In another example, the processor may detect the need for the change to a setting of a parameter for a volume of air exchange per respiration cycle. In yet another example, the processor may detect the need for the change to a setting of a parameter for selecting whether the artificial respiratory device forces air into a lung of the user or applies a positive air pressure to assist the user by keeping the airways open while the user is trying to breathe. When the need for the change to the setting of the parameter of the artificial respiratory device of the user is detected, the processor proceeds to step 240. Otherwise, the processor proceeds to step 299. Again, in one embodiment, a different threshold is set for each individual patient as to the need to change a setting, e.g., a change of forty-five degrees in a bed setting may cause a setting of the artificial respiratory device to be changed for one patient but not for another patient.

In step 240, the processor changes the setting of the parameter for which the need for the change to the setting of the parameter is detected in accordance with the activity level of the user. For the example described above, the processor may change the setting of the parameter on the artificial respiratory device of the user to a setting that is appropriate when the user is sitting at an angle of sixty degrees. For instance, the setting of the parameter of the artificial respiratory device may be changed for performing at least one of: increasing a number of respiration cycles per minute, decreasing a number of respiration cycles per minute, increasing a volume of air exchange per respiration cycle, decreasing a volume of air exchange per respiration cycle, forcing air into a lung of the user, and applying a positive air pressure to assist the user while the user is breathing.

In optional step 250, the processor records the setting of the parameter that is changed. For example, the processor stores the setting of the parameter that is changed in a database, e.g., database 113.

In optional step 260, the processor provides a notification that comprises the setting of the parameter that is changed. For example, the processor may provide a notification to at least one of: a system of a healthcare service provider, a local device, a server that performs a remote function, a system of a caregiver, a system of a family member, and a system of a next of kin of the user. The remote function may be monitoring of the artificial respiratory device, scheduling a repair and/or maintenance for the artificial respiratory device, scheduling a delivery of a supply that is to be used in conjunction with the artificial respiratory device, and so on. Furthermore, method 200 may further optionally allow (not shown) a medical staff to provide inputs as to the changing of a setting, e.g., the medical staff may not agree with the changing of the setting or may simply provide a different setting change and so on. The processor then proceeds either to step 210 or step 299 to end the process.

In addition, although not specifically specified, one or more steps, functions or operations of method 200 may include a storing, displaying and/or outputting step as required for a particular application. In other words, any data, records, fields, and/or intermediate results discussed in the method can be stored, displayed and/or outputted either on the device executing the method or to another device, as required for a particular application.

Furthermore, steps, blocks, functions or operations in FIG. 2 that recite a determining operation or involve a decision do not necessarily require that both branches of the determining operation be practiced. In other words, one of the branches of the determining operation can be deemed as an optional step. Moreover, steps, blocks, functions or operations of the above described method 200 can be combined, separated, and/or performed in a different order from that described above, without departing from the example embodiments of the present disclosure.

As such, the present disclosure provides at least one advancement in the technical field of controlling an artificial respiratory device. For instance, in one example, the present disclosure detects a change in an activity level of a user based on data that is gathered from at least one sensor that is located in a vicinity of a user, detects a change to a setting of at least one parameter of an artificial respiratory device of the user responsive to the change in the activity level of the user, and changes the setting of the parameter on the artificial respiratory device of the user in accordance with the activity level of the user. The artificial respiratory device of the user may then be timely controlled by the application server of the network service provider based on the activity level of the user. In other words, the parameters on the artificial respiratory device of the user may be dynamically controlled by the application server of the network service provider to quickly respond to the need of the user. The artificial respiratory device is situated at a remote location, e.g., a room at a hospital or a nursing home, while the application server is situated at a location of the network service provider. Thus, the application server is remotely controlling the parameters of the artificial respiratory device.

In one embodiment, the service provided by the network service provider can be provided to hospital and nursing home operators. In other words, the hospital and nursing home operators may subscribe to a service where the monitoring and operation of one or more artificial respiratory devices can be remotely performed by on a platform operated by the network service provider. This allows the hospital and nursing home operators to leverage the reliable network infrastructure of the network service provider without having to build its own network infrastructure. It also addresses an efficiency issue where a centralized monitoring system can be provided so that a plurality of locations (e.g., different rooms within a single hospital or even rooms in different hospitals can be all centrally monitored with a small medical staff receiving the notifications as discussed above.

FIG. 3 depicts a high-level block diagram of a computer suitable for use in performing the functions described herein. As depicted in FIG. 3, the system 300 comprises one or more hardware processor elements 302 (e.g., a central processing unit (CPU), a microprocessor, or a multi-core processor), a memory 304, e.g., random access memory (RAM) and/or read only memory (ROM), a module 305 for controlling an artificial respiratory device, and various input/output devices 306 (e.g., storage devices, including but not limited to, a tape drive, a floppy drive, a hard disk drive or a compact disk drive, a receiver, a transmitter, a speaker, a display, a speech synthesizer, an output port, an input port and a user input device (such as a keyboard, a keypad, a mouse, a microphone and the like)). Although only one processor element is shown, it should be noted that the computer may employ a plurality of processor elements. Furthermore, although only one computer is shown in the figure, if the method 200 as discussed above is implemented in a distributed or parallel manner for a particular illustrative example, i.e., the steps of the above method 200 or the entire method 200 is implemented across multiple or parallel computers, then the computer of this figure is intended to represent each of those multiple computers.

Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented.

It should be noted that the present disclosure can be implemented in software and/or in a combination of software and hardware, e.g., using application specific integrated circuits (ASIC), a programmable gate array (PGA) including a Field PGA, or a state machine deployed on a hardware device, a computer or any other hardware equivalents, e.g., computer readable instructions pertaining to the method(s) discussed above can be used to configure a hardware processor to perform the steps, functions and/or operations of the above disclosed method.

In one embodiment, instructions and data for the present module or process 305 for controlling an artificial respiratory device (e.g., a software program comprising computer-executable instructions) can be loaded into memory 304 and executed by hardware processor element 302 to implement the steps, functions or operations as discussed above in connection with the illustrative method 200. Furthermore, when a hardware processor executes instructions to perform “operations,” this could include the hardware processor performing the operations directly and/or facilitating, directing, or cooperating with another hardware device or component (e.g., a co-processor and the like) to perform the operations.

The processor executing the computer readable or software instructions relating to the above described method can be perceived as a programmed processor or a specialized processor. As such, the present module 305 for controlling an artificial respiratory device (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. Furthermore, a “tangible” computer-readable storage device or medium comprises a physical device, a hardware device, or a device that is discernible by the touch. More specifically, the computer-readable storage device may comprise any physical devices that provide the ability to store information such as data and/or instructions to be accessed by a processor or a computing device such as a computer or an application server.

While various embodiments have been described above, it should be understood that they have been presented by way of example only, and not a limitation. Thus, the breadth and scope of a preferred embodiment should not be limited by any of the above-described exemplary embodiments, but should be defined only in accordance with the following claims and their equivalents.

What is claimed is:

1. An apparatus comprising:
a processor of a communications network; and
a computer-readable storage device storing a plurality of instructions which, when executed by the processor, cause the processor to perform operations, the operations comprising:
detecting a change in an activity level of a user based on data that is gathered from at least one sensor that is located in a vicinity of the user;
detecting a need for a change to a setting of at least one parameter of an artificial respiratory device associated with the user responsive to the change in the activity level of the user being detected; and
changing the setting of the at least one parameter of the artificial respiratory device in accordance with the activity level of the user.

2. The apparatus of claim 1, wherein the change in the activity level of the user is detected when the change in the activity level of the user reaches or exceeds a predetermined threshold for the change in the activity level of the user, wherein the predetermined threshold for the change in the activity level of the user is based on a profile of the user.

3. The apparatus of claim 1, wherein the at least one parameter comprises at least one of: a parameter for a number of respiration cycles per minute, a parameter for a volume of air exchange per respiration cycle, and a parameter for selecting between the artificial respiratory device forcing air into a lung of the user or applying a positive air pressure to assist the user.

4. The apparatus of claim 1, wherein the setting of the at least one parameter is changed for performing at least one of: increasing a number of respiration cycles per minute, decreasing a number of respiration cycles per minute, increasing of a volume of air exchange per respiration cycle, decreasing a volume of air exchange per respiration cycle, forcing air into a lung of the user, and applying a positive air pressure to assist the user while the user is breathing.

5. The apparatus of claim 1, the operations further comprising:
recording the setting of the at least one parameter that is changed.

6. The apparatus of claim 1, the operations further comprising:
providing a notification, wherein the notification comprises the setting of the at least one parameter that is changed.

7. The apparatus of claim 6, wherein the notification is provided to at least one of: a system of a healthcare service provider, a local device, a server that performs a remote function, a system of a caregiver, a system of a family member, and a system of a next of kin of the user.

8. The apparatus of claim 7, wherein the remote function comprises at least one of: monitoring of the artificial respiratory device, scheduling a repair or maintenance for the artificial respiratory device, and scheduling a delivery of a supply that is to be used in conjunction with the artificial respiratory device.

9. The apparatus of claim 1, wherein the data is gathered from the at least one sensor directly.

10. The apparatus of claim 1, wherein the data is gathered from the at least one sensor via a local device.

11. The apparatus of claim 10, wherein the local device receives the data from the at least one sensor in accordance with a predetermined schedule for receiving data from the at least one sensor.

12. The apparatus of claim 10, wherein the local device provides the data to an application server of a network service provider operating the communications network in accordance with a predetermined schedule for providing the data to the application server.

13. The apparatus of claim 1, wherein the setting of the at least one parameter is changed by selecting a new setting for the at least one parameter from a set of allowable settings predefined for the user.

14. The apparatus of claim 13, wherein the new setting is selected in part based on the change in the activity level of the user from a previous activity level of the user.

15. The apparatus of claim 1, wherein the setting of the at least one parameter is changed on a continuous basis with a periodic time period.

16. The apparatus of claim 1, wherein the changing of the setting of the at least one parameter is constricted to a predefined range based on a profile of the user.

17. The apparatus of claim 1, wherein the artificial respiratory device is a continuous positive airway pressure machine.

18. The apparatus of claim 1, wherein the at least one sensor comprises at least one of: a biometric sensor, a motion sensor, and a sensor for sensing a user action, wherein the user action is an action performed by the user.

19. A non-transitory computer-readable storage device storing a plurality of instructions which, when executed by a processor of a communications network, cause the processor to perform operations, the operations comprising:
detecting a change in an activity level of a user based on data that is gathered from at least one sensor that is located in a vicinity of the user;
detecting a need for a change to a setting of at least one parameter of an artificial respiratory device associated with the user responsive to the change in the activity level of the user being detected; and
changing the setting of the at least one parameter of the artificial respiratory device in accordance with the activity level of the user.

20. A method comprising:
detecting, by a processor, a change in an activity level of a user based on data that is gathered from at least one sensor that is located in a vicinity of the user;
detecting, by the processor, a need for a change to a setting of at least one parameter of an artificial respiratory device associated with the user responsive to the change in the activity level of the user being detected; and
changing, by the processor, the setting of the at least one parameter of the artificial respiratory device in accordance with the activity level of the user.
